(19) United States
(12) Patent Application Publication
(10) Pub. No.: US 2009/0074693 A1
(43) Pub. Date: Mar. 19, 2009

(54) TEMPORARY HAIR COLORING COMPOSITION AND METHOD OF APPLICATION

(76) Inventor: Cindy Mace, Holtsville, NY (US)

Correspondence Address: ANTHONY R. BARKUME, 20 GATEWAY LANE, MANORVILLE, NY, 11949, US

(21) Appl. No.: 11/854,824

(22) Filed: Sep. 13, 2007

(51) Int. Cl. A61K 8/25; A61K 8/27; A61Q 5/06

(52) U.S. Cl. 424/70.6

(57) ABSTRACT

A method of temporarily altering the color of a person's hair to a desired color. First, a hair coloring mixture is prepared, which comprises a facial skin coloring powder, eyeshadow, and talc, wherein the facial skin coloring powder has a color substantially equivalent to the desired color. The hair coloring mixture is then to a person's hair in the desired spot, preferably with a natural hair applicator brush such as a boar's hair brush. The facial skin coloring powder is preferably a microfine loose powder with approximately ⅛ part of facial skin coloring powder, ⅛ part of eyeshadow, and ¾ part of talc.

### BACKGROUND OF THE INVENTION

The present invention relates to a composition for use in temporarily coloring human hair and the application of the composition.

People, both male and female, often desire to change the color of their hair. Products exist, such as hair dyes, which may be applied to the entirety of the hair in order to effect such change. Products also exist that allow the user to apply highlights to the hair by coloring part of the hair but not the entire head. In either case, application of dyes is a time consuming, messy and expensive procedure.

People also often desire to be able to change the color of their hair in certain defined regions of their head, in particular where there are regions of grey hair in an otherwise black or dark brown head of hair. These people may want to “touch-up” the grey hair to appear to be the same or substantially the same color as the rest of their hair. Products such as hair dyes are not useful for this situation because they do not lend themselves to touching up a defined region of hair, but are instead intended to be applied to the entire head of hair.

It is therefore desired to provide for a temporary hair coloring substance and method of application that is relatively easy, that can be done in any location (not just at a salon), and that can be done by the person himself or herself without requiring someone else to apply the product.

### SUMMARY OF THE INVENTION

Thus, the present invention is a method of temporarily altering the color of a person's hair to a desired color. First, a hair coloring mixture is prepared, which comprises a facial skin coloring powder, eyeshadow, and talc, wherein the facial skin coloring powder has a color substantially equivalent to the desired color. The hair coloring mixture is then applied to a person's hair in the desired spot, preferably with a natural hair applicator brush such as a boar's hair brush.

The facial skin coloring powder is preferably a microfine loose powder with approximately ⅛ part of facial skin coloring powder, ⅛ part of eyeshadow, and ¾ part of talc, which was determined experimentally as set forth herein.

The present invention does not replace permanent hair color, but is a solution to the gray or natural hair when the hair color is growing out. The hair coloring mixture lays on top of and adheres to the hair shaft and gives the illusion of permanent color (i.e. it is a hair makeup).

### BRIEF DESCRIPTION OF THE DRAWING

FIG. 1 is an illustration of an applicator brush used with the present invention.

### DETAILED DESCRIPTION OF THE PREFERRED EMBODIMENT

As will be described in detail herein, the present invention allows a person to apply a touch up application to defined areas of their hair and/or scalp and effect a temporary color change in a convenient and quick manner, without requiring a professional application of a dye as in the prior art.

A hair coloring mixture is first prepared, which will include a facial skin coloring powder, eyeshadow, and talc. The facial skin coloring power is selected based on the desired color, such as dark brown, auburn, black, etc. For example, a product known as microfine loose powder, marketed by LadyBird Cosmetics, has been utilized with success in this invention. This microfine loose powder is otherwise used as a facial application for blending skin tone, but it has been discovered by the applicant that the combination of this powder with talc and eye shadow and applied as described herein leads to the present invention for touching up the hair and/or scalp of a person as described herein.

The hair coloring mixture also includes a quantity of talc. Talc is used in the present invention in order to increase the adherence of the mixture to the natural oils of the hair, thus enabling the mixture to be used in this hair coloring application when dry. Although some talc may be found in the original microfine loose powder formulation, it is understood that this amount of talc is intentionally kept at a relatively small amount since the presence of talc will cause an opacity that is undesired in the intended application of the microfine loose powder, which is for blending skin tone and not for covering the skin. Thus, the addition of talc in the present invention provides two benefits. First, the addition of talc will increase the opacity of the microfine loose powder which although is undesirable in the prior art application of skin is actually desirable in the present invention application to hair and/or scalp. Second, the addition of talc will increase the adherence of the resulting mixture to the oils of the hair, thus causing the mixture to remain applied to the person for a longer period of time. The mixture will also adhere to the hair when a moderate amount of moisture or wetness is present, such as in a rain shower. An excessive amount of water, such as when the person is taking a shower and shampooing, will cause the powder mixture to be washed out, which is usually a desirable effect. In this case, the person would need to reapply the mixture as part of the grooming process after the shower is completed.

It is understood that the microfine loose powder is initially composed of talc (in an amount inadequate to allow the present invention to function), zinc stearate, kaolin, methylparaben, propylparaben, ethylparaben, and trisodium EDTA. Various coloring agents are also in the powder, such as mica, titanium dioxide, carmine, ferric ferrocyanide, iron oxides, ultramarines, chromium oxide greens, chromium hydroxide green, manganese violet, yellow 5 lake, and blue 1 lake. An additional amount of talc is added in order to increase the opacity of the mixture, to increase the adherence of the mixture to the hair and/or scalp, and to improve on the consistency of the mixture accordingly. It is cautioned however that the addition of too large a quantity of talc will change the color of the mixture which may not be desired.

As set forth herein, the original set of mixtures did not provide optimal results, so a quantity of matte eyeshadow was added to the mixture, yielding the desired results.

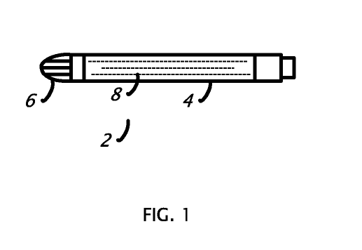

Application of the mixture as described above to targeted areas of the person's hair and/or scalp is accomplished in the present invention by the use of a natural hair applicator brush, preferably a boar's hair applicator brush 2 as illustrated in FIG. 1. The hair coloring mixture 8 is placed into the powder containment portion 4 so and is applied to the person's hair via the brush portion 6. It has been found that this type of applicator is optimal since it gives the user control over the specific area of application without requiring the help of another person. For example, an attempt was made to use a shaker container and shake the mixture over the target area, but that did not give the user the control provided by the spot applicator described herein.

Thus, by gradually applying the powder mixture with the boar's hair applicator as shown, the user is able to apply the mixture as desired on the target location, which is usually a grey spot or root(s) in the person's hair. This will help prolong the time that a person may have between professional coloring applications, if desired.

In addition to providing spot coloring of a person's hair, the present invention has been found to equally beneficial to application to an area of a person's scalp. That is, a person may have a thinning area of his head and wish to fill it in with the powder of the present invention. The powder may also be applied to an area that is a combination of grey hair and thinning hair.

Several experiments were conducted in order to determine a preferable mixture of the microfine loose power and talc (without the matte eyeshadow), as shown below:

### Sample 1

A mixture was created of ½ part microfine loose powder and ½ part talc. This mixture was applied with the boar's hair applicator, The results were an excessively white color and a clogged applicator.

### Sample 2

A mixture was created of ¾ part microfine loose powder and ¼ part talc. This mixture was applied with the boar's hair applicator. The results were a mixture that was too dry and could not be applied easily for a uniform coverage.

### Sample 3

A mixture was created of ¼ part microfine loose powder and ¾ part talc. This mixture was applied with the boar's hair applicator. The results were that the mixture was still too chalky and did not apply easily.

We then added a portion of matte eyeshadow to the mixture:

### Sample 4

A mixture was created of ⅛ part microfine loose powder, ⅛ matte eyeshadow and ¾ part talc. This mixture was applied with the boar's hair applicator. This mixture provided the correct coloring and an appropriate texture for the intended application. This is therefore the preferable mixture based on the above experimentation.

I claim:

1. A method of temporarily altering the color of a person's hair to a desired color comprising the steps of:
a. preparing a hair coloring mixture comprising a facial skin coloring powder, eyeshadow, and talc, the facial skin coloring powder having a color substantially equivalent to the desired color; and
b. applying the hair coloring mixture to a person's hair.

2. The method of claim 1 wherein the facial skin coloring powder is a microfine loose powder.

3. The method of claim 1 wherein the applying step comprises the step of applying the hair coloring mixture to a person's hair with a natural hair applicator brush.

4. The method of claim 3 wherein the natural hair applicator brush comprises boar's hair.

5. The method of claim 3 wherein the natural hair applicator comprises a powder containment area and a brush portion in proximity thereto, wherein the hair coloring mixture is placed into the powder containment area so and is applied to the person's hair via the brush portion.

6. The method of claim 1 wherein the hair coloring mixture comprises approximately ⅛ part of facial skin coloring powder, ⅛ part of eyeshadow, and ¾ part of talc.

7. The method of claim 1 further comprising the step of
c. applying the hair coloring mixture to an area of scalp in close proximity to the person's hair.

8. The method of claim 1 wherein the facial skin coloring powder comprises talc, zinc stearate, kaolin, methylparaben, propylparaben, ethylparaben, and trisodium EDTA.

9. A hair coloring mixture for temporarily altering the color of a person's hair to a desired color, comprising a facial skin coloring powder, eyeshadow, and talc, the facial skin coloring powder having a color substantially equivalent to the desired color.

10. The hair coloring mixture of claim 9 wherein the facial skin coloring powder is a microfine loose powder.

11. The hair coloring mixture of claim 9, comprising approximately ⅛ part of facial skin coloring powder, ⅛ part of eyeshadow, and ¾ part of talc.

12. The hair coloring mixture of claim 9, wherein the facial skin coloring powder comprises talc, zinc stearate, kaolin, methylparaben, propylparaben, ethylparaben, and trisodium EDTA.
